(19) United States
(12) Patent Application Publication
(10) Pub. No.: US 2026/0157841 A1
(43) Pub. Date: Jun. 11, 2026

(54) IMPLANT FOR AN AREAL TREATMENT OF A BONE DEFECT

(71) Applicant: Karl Leibinger Asset Management GmbH & Co. KG, Mühlheim an der Donau (DE)

(72) Inventors: Adem Aksu, VS-Schwenningen (DE); Lena Schaarschmidt, Aldingen (DE)

(21) Appl. No.: 19/416,330

(22) Filed: Dec. 11, 2025

(30) Foreign Application Priority Data
Dec. 11, 2024 (DE) 102024137111.0

(51) Int. Cl. A61F 2/00; A61F 2/28; B33Y 10/00

(52) U.S. Cl. CPC A61F 2/0063; A61F 2/2846; B33Y 10/00; A61F 2002/0068; A61F 2240/001; A61F 2250/0024; A61F 2310/00023

(57) ABSTRACT

An implant (I) for an areal treatment of a bone defect includes a lattice structure (GS) having a top side (OS) and an underside (US) facing away from the top side (OS) and on which the implant (I) is fixable to bone. The lattice structure (GS) has at least one support section (TA) produced by an additive manufacturing process. In addition, a cover layer (DS) is applied on each of at least some portions of the at least one support section (TA). The cover layer has a lower hardness compared to the at least one support section (TA).

### CROSS-REFERENCE TO RELATED APPLICATION

The present application is related and has right of priority to German Patent Application No. DE102024137111.0 filed on Dec. 11, 2024, which is incorporated by reference in its entirety for all purposes.

### TECHNICAL FIELD

The invention relates generally to an implant for an areal treatment of a bone defect, in particular a bone defect in the area of the thorax or the cranium.

### BACKGROUND

In the field of implants, variants are known that are used for the areal treatment of bone defects. Such an implant frequently has a lattice structure, which is at least partially flexible and thus enables the implant to be adapted to different shapes. Implants designed in this way can be used in the thorax area and, in particular, in place of rib plates in the case of rib fractures, so that, by being arranged over a large area on the thorax, areas between different ribs are also covered. Due to the lattice structure, internal organs are protected while, at the same time, sufficient flexibility is achieved for enabling sufficient mobility of the thorax.

Furthermore, implants having a lattice structure in the area of the cranium are provided for treating skull defects, wherein the lattice structure enables the implant to be adapted to curvatures of the skull and additionally ensures sufficient elasticity. An implant for the areal treatment of bone defects typically has a mesh-like support section, which forms the lattice structure and is fixated to the bone and used for stabilization.

DE 197 46 396 A1 describes an implant provided for an areal treatment of bone defects in the area of the cranium. The implant has a lattice structure, which is composed of a plurality of segments which are closed and connected to one another. The lattice structure can be fastened at an underside in the corresponding area of the cranium for fixing bone portions or for bridging bone defects, wherein the fastening can be carried out by guiding a bone screw through each individual segment. The interconnected segments form a continuous support section in the lattice structure, via which the bone portions and the bone defect can be stabilized. The lattice structure is produced by etching titanium.

### BRIEF SUMMARY

Example aspects of the present invention provide an implant for an areal treatment of bone defects, wherein complex geometries of the implant are to be achievable with low manufacturing outlay. Furthermore, the implant according to example aspects of the invention is intended to minimize irritations, in particular tissue irritations, in the body of the patient.

According to example aspects of the invention, an implant includes a flexible lattice structure having a top side and an underside facing away from the top side and at which the implant is to be fixated to bone. The lattice structure has at least one support section.

The implant according to example aspects of the invention is provided for an areal treatment of a bone defect, wherein this is, in particular, a bone defect in the area of the thorax or the cranium. For this areal treatment, the implant is equipped with a lattice structure which is preferably flat. The lattice structure has a top side and an underside, which face away from one another and of which the underside is the side with which the implant is placed and fixated facing the bone defect.

An “areal” treatment is to be understood within the meaning of the invention, in particular, to mean that a treatment area is covered by the implant when used on the bone defect. Within the framework of example aspects of the invention, the implant can be specifically provided for use in this areal treatment to connect bone segments of a fractured bone and/or to establish connections between different bones and/or to cover bone defects. A treatment area to be covered by the implant according to example aspects of the invention can range from a few square millimeters to many square centimeters depending on the specific use case, i.e., in particular whether an application is intended in the area of the thorax or the cranium.

The lattice structure is flexible, whereby the lattice structure is given 3-dimensional deformability in order to be able to easily adapt the shape of the implant to the particular treatment area. The lattice structure of the implant according to example aspects of the invention is thus adaptable, due to its flexible design, to curved bone defects and to bone defects having an irregular shape. Furthermore, the lattice structure can be designed within the framework of example aspects of the invention to be adapted by the attending surgeon in terms of its size and shape by removing parts of the lattice structure, for example by cutting the lattice structure to size.

According to example aspects of the invention, the lattice structure has at least one support section which is preferably used to stabilize bone. In particular, this at least one support section is intended to be fixated to the bone in the region of the bone defect to be treated, i.e. connected to a plurality of bone segments of a bone or to one or more bones or bone portions. The stabilization via the at least one support section is achieved, in particular, in that the stabilization establishes one or more connections to bone in the applied state of the implant. Preferably, the at least one support section is provided with at least one fixation point for this purpose, at which the fixation of the implant is achievable, for example, using a respective bone screw. The implant according to example aspects of the invention can have one or more support sections.

Example aspects of the invention include the at least one support section produced by an additive manufacturing process. Moreover, a cover layer is at least partially applied on the at least one support section, which cover layer has a lower hardness compared to the at least one support section. In other words, in the implant according to example aspects of the invention, the at least one support section has been formed within the framework of an additive manufacturing process. In addition, the at least one support section is at least partially coated with a cover layer, wherein this cover layer has a lower hardness compared to the at least one support section.

Such a design of an implant has the advantage that the at least one support section can be easily designed having complex geometries due to the additive manufacturing and this is possible with low manufacturing outlay. This makes it possible to obtain, in particular, a complex and simultaneously flexible structure of the at least one support section and, thus, of the lattice structure. Due to the at least partial application of the cover layer on the at least one support section, and the lower hardness of this cover layer compared to the support section, skin and/or tissue irritations can be avoided, which are caused by the greater hardness of the at least one support section and, in particular, due to production-related irregularities in the at least one support section. This is the case because, due to the at least partial coverage of the at least one support section with the cover layer, a softer surface of the implant is obtained in the particular area due to the lower hardness of the cover layer. Overall, an implant is therefore obtained, in which at least one support section of a lattice structure is designed in a suitable manner with low manufacturing outlay and, when used, the occurrence of skin and/or tissue irritations can be substantially reduced.

Example aspects of the invention include that the at least one support section of the implant according to example aspects of the invention has been formed by an additive manufacturing process, wherein this is particularly preferably carried out within the framework of a 3D printing method. Furthermore, the at least one support section is provided with the cover layer at least on subsections thereof, which cover layer is less hard compared to the at least one support section and thus is softer. As a result, the implant according to example aspects of the invention is provided with a softer surface in the region of the coating of the least one support section with the cover layer.

Within the meaning of the invention, “hardness” is to be understood as the mechanical resistance applied against mechanical penetration. Accordingly, each respective cover layer applies a lower mechanical resistance to a mechanical penetration than is the case with the at least one support section. Each respective cover layer can be described as softer compared to the at least one support section.

According to example aspects of the invention, the at least one support section is at least partially coated with the cover layer, i.e, the at least one support section can therefore be provided with the cover layer on one or more portions thereof or also around the entire periphery thereof.

According to one possible example embodiment, at least one subsection of the at least one support section on the top side of the lattice structure is coated with the cover layer. In this case, the cover layer is therefore applied on some portions of the at least one support section on the top side, whereby some portions of the at least one support section on the top side are uncoated. Alternatively, the at least one support section is completely coated with the cover layer on the top side of the lattice structure, such that the at least one support section is completely coated with the cover layer on the top side. Advantageously, the at least one support section is therefore either covered in certain regions in a targeted manner or completely covered on the top side. In the latter case, tissue irritations are thereby completely prevented on the top side, whereas, with the coating in some portions, contact areas with tissue and/or irregularities of the support section, for example edges or the like, can be intentionally designed to be softer with the aid of the cover layer.

Alternatively or additionally, the at least one support section can be coated with the cover layer on at least one subsection on the underside of the lattice structure. Some portions of the at least one support section are provided with the cover layer on the underside of the lattice structure in this case, whereby portions of the at least one support section on the underside are uncoated. However, the at least one support section could also be completely coated with the cover layer on the underside of the lattice structure, whereby the at least one support section is then completely covered on the underside with the cover layer. In both cases, a softer surface is obtained on at least some portions of the underside of the implant by either partially or completely coating the at least one support section on the underside with each respective cover layer. As a result, tissue irritations on the underside of the implant can be reduced. In the case of the partial coating of the at least one support section, this is coated in particular intentionally in certain contact regions with tissue and/or at irregularities, for example edges or the like.

The aforementioned examples aspects of the invention can be achieved alternatively or additionally, whereby the at least one support section can be coated partially or completely on the top side, partially or completely on the underside, or both partially or completely on the top side and partially or completely on the underside.

In combination, an example design of the implant is also conceivable in which the at least one support section is completely encapsulated in each respective cover layer. In this case, the at least one support section is then therefore completely surrounded by the cover layer and thus completely coated.

According to one possible example embodiment of the invention, the at least one support section has a mesh structure, which is formed by closed segments which are connected to one another via intermediate segments. As a result, a suitable design is achieved, in which a high degree of mobility and thus flexibility can be achieved in the region of the at least one support section. Preferably, the closed segments are ring-shaped, wherein the segments can also have shapes deviating therefrom, for example the shape of a polygon. The segments are connected to one another in the mesh structure via intermediate segments, which can be linear or non-linear. It would also be conceivable that the intermediate segments each define a closed segment by being fastened to one another.

Within the framework of example aspects of the invention, the at least one support section can be plate-shaped. In this case, the at least one support section has the shape of a plate, wherein the at least one support section can be designed as a rigid plate. In the implant according to example aspects of the invention, a rigid region can thereby be intentionally defined, in which particularly high stability of the implant is to be achieved. The plate-shaped design is obtained, in particular, when the implant according to example aspects of the invention is composed of a plurality of support sections, wherein one or more of the support sections then each has a mesh structure and one or more of the support sections can be designed in the shape of a plate.

In a further possible example embodiment of the invention, the cover layer is porous. This has the advantage that, due to this porous design, a particularly low hardness of the respective cover layer and thus also a very soft surface can be obtained. On the other hand, the possibility is thereby created that tissue can grow into the implant and vascularization is possible. In addition, bodily fluids can thereby pass through the cover layer. In a development of this example embodiment, the at least one support section is at least partially embedded into the porous cover layer. Advantageously, as a result, an even, soft surface can be obtained in the corresponding area.

Alternatively, the cover layer can be non-porous. Thus, a very smooth and simultaneously soft surface can be achieved in this area, whereby tissue irritations can be largely avoided. Due to the non-porous design of each respective cover layer, tissue can be prevented from growing thereon. In particular, a structuring of the cover layer corresponds to a structuring which the at least one support section has in the region of the coating with each respective cover layer. This has the advantage that the structure of the at least one support section is retained toward the outside.

Preferably, the at least one support section is formed of a first biocompatible material. The cover layer is preferably made of a second biocompatible material, which has a lower hardness than the first material. As a result, the hardness of the cover layer, which is lower compared to the at least one support section, can be achieved in a simple way. In addition, due to a suitable selection of the second material, further suitable properties can be achieved on the surface, for example the formation of a particularly smooth surface when machining the material.

In a development of the aforementioned possible example embodiment, the first material is a metal or a metal alloy, in particular titanium or a titanium alloy. Most particularly preferably, however, the first material is a plastic, wherein this plastic is, in particular, a polymer, preferably a thermoplastic, and, particularly preferably, polyether ether ketone (PEEK). This is the case because polyether ether ketone is distinguished by very good biocompatibility and a high achievable strength.

The second material is preferably a plastic, in particular a polymer, preferably a thermoplastic and, particularly preferably, polyethylene (PE), for example ultra high molecular weight polyethylene (UHMWPE) or high density polyethylene (HDPE). As a result, the cover layer, which is softer compared to the at least one support section, can be reliably produced. Furthermore, a porous cover layer can thus be obtained by using PE granules, and a non-porous cover layer can be obtained by using PE powder, wherein in the latter case a particularly smooth surface can be obtained.

Most particularly preferably, the two aforementioned example variants are jointly implemented, wherein, in order to produce the implant according to example aspects of the invention, the additive manufacturing of the at least one support section made of polyether ether ketone (PEEK) is initially carried out. Then, for cleaning purposes, a plasma treatment/plasma activation of the at least one support section is preferably carried out before the at least one support section is placed or embedded together with the PE powder or PE granules layer-by-layer in a negative mold. In a subsequent pressing process, the polyethylene (PE) is then heated together with the polyether ether ketone (PEEK), whereby the polyethylene is fused with the polyether ether ketone. As a result, a loadable bond between polyether ether ketone and polyethylene can be obtained.

Within the framework of example aspects of the invention, the at least one support section and each respective applied cover layer could also consist of one and the same material. Thus, the at least one support section and the cover layer could each be made, for example, of polyethylene.

According to one example embodiment of the invention, the lattice structure has one single continuous support section. In this case, the lattice structure is therefore formed by one single support section.

Alternatively, the lattice structure has a plurality of support sections, wherein adjacent support sections are connected to one another via a respective intermediate connecting section, which has a lower rigidity compared to the adjacent support sections. This has the advantage that the flexibility of the lattice structure can thereby be intentionally increased in the region of the respective connecting section. Due to the lower rigidity of each respective intermediate connecting section, the lattice structure and thus the implant can be more easily deformed in this area. In addition, as a result, the risk of skin and/or tissue irritations can also be intentionally reduced locally by the at least one intermediate connecting section.

Particularly preferably, the lower rigidity of each respective intermediate connecting section is obtained in that each respective intermediate connecting section is made of a biocompatible material having a lower material rigidity than the material of the at least one support section. When the at least one support section and the cover layer are made of different materials, each respective intermediate connecting section is made, in particular, of the same material as the cover layer.

Within the framework of example aspects of the invention, each respective intermediate connecting section can be present in the form of a linear or non-linear intermediate piece or in the form of a mesh structure. In addition, each respective intermediate connecting section could also be plate-shaped.

Alternatively or additionally, the respective support section and each respective connecting section are integrally joined to one another. This is achieved, in particular, when each respective connecting section and the respective support section if formed of a plastic material and, in particular, a thermoplastic plastic.

In order to design the connection between the respective support section and each respective connecting section to be more robust, it is conceivable within the framework of example aspects of the invention that the respective support section and each respective connecting section overlap in the region of the respective fixation in a direction extending transversely to the top side and the underside. Thus, the load-bearing capacity of the connection between the respective support section and each respective connecting section is increased. Most particularly preferably, each respective connecting section engages around the respective support section in the region of the respective fixation on each side with protruding connecting segments.

### BRIEF DESCRIPTION OF THE DRAWINGS

Advantageous embodiments of the invention, which are explained in the following, are shown in the drawings. In the drawings:

FIG. 1 shows a top view of an implant corresponding to a first example embodiment;

FIG. 2 shows a schematic sectional view of the implant from FIG. 1;

FIG. 3 shows a perspective view of an implant according to a second possible example embodiment;

FIGS. 4 through 6 show schematic views of possible example modifications of the implants from FIGS. 1 and 3:

FIG. 7 shows a schematic view of an implant according to a third example embodiment;

FIG. 8 shows a schematic sectional view of the implant from FIG. 7; and

FIGS. 9 and 10 show partial views of implants corresponding to further possible example embodiments.

### DETAILED DESCRIPTION

Reference will now be made to embodiments of the invention, one or more examples of which are shown in the drawings. Each embodiment is provided by way of explanation of the invention, and not as a limitation of the invention. For example, features illustrated or described as part of one embodiment can be combined with another embodiment to yield still another embodiment. It is intended that the present invention include these and other modifications and variations to the embodiments described herein.

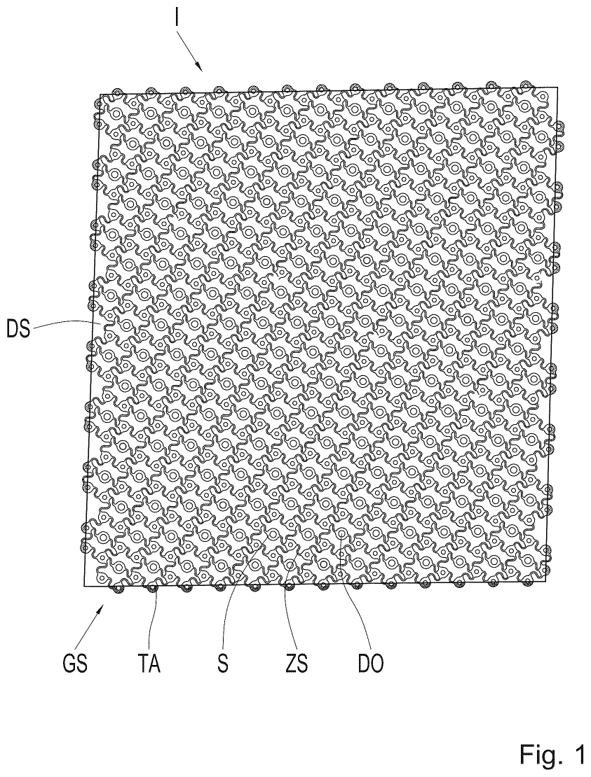

FIG. 1 shows a top view of an implant I provided for an areal treatment of bone defects and, in particular, in the thorax area. The implant I includes a lattice structure GS, which is formed by a support section TA in the present case. As is apparent in FIG. 1, this support section TA has a mesh structure, in that the support section TA is composed of ring-shaped segments S and intermediate segments ZS via which the ring-shaped segments S are connected to one another. The intermediate segments ZS extend linearly.

In the support section TA, the ring-shaped segments S each form a through-hole DO, in each of which a bone screw for fixating the implant I can be received, wherein the implant I can be fixated to bone in the region of the bone defect to be treated by bone screws which have been guided through. Thus, with the aid of the support section TA, bone segments or bone portions of one or more rib bones, and/or a plurality of rib bones can be connected to one another in the thorax area, wherein the support section TA stabilizes the bone segments or bone portions with respect to one another, and/or the rib bones with respect to one another.

The intermediate segments ZS allow the ring-shaped segments S to move relative to one another, whereby the overall mesh structure of the support section TA is flexible. This gives the lattice structure GS of the overall implant I flexible properties and enables, on the one hand, adaptation to curvatures of the bones in the region of the bone defect to be treated. On the other hand, when the implant I is in the fixated state, a certain mobility is thereby permitted in the region of the bone defect to be treated, in order to enable the thorax to move due to the breathing or movement of the patient.

The support section TA has been made of polyether ether ketone (PEEK) within the framework of an additive manufacturing process in the present example case, wherein the support section TA has been specifically shaped in the 3D printing process. As a result, the complex mesh structure of the support section TA can be produced with low manufacturing outlay, wherein high stability of the support section TA can also be achieved via the biocompatible material polyether ether ketone (PEEK).

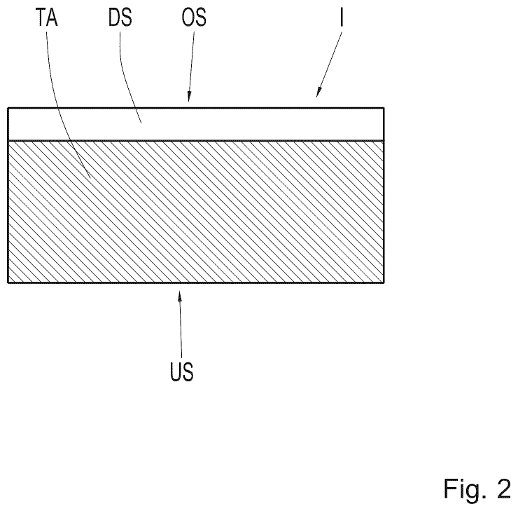

As a result of the additive manufacturing of the support section TA, however, a raw surface and, in part, also hard edges on the support section TA are produced, which can result in corresponding tissue irritations once the implant has been placed in the body of the particular patient. Furthermore, the mesh structure of the support section TA is noticeable through the tissue or the skin of the patient under some circumstances, which can also result in corresponding irritations. In order to reduce the risk of such irritations, the support section TA is partially provided with a cover layer DS, which has a lower hardness compared to the support section TA and, thus, is softer. As is apparent when FIG. 1 is considered in combination with the schematic sectional view in FIG. 2, this cover layer DS is applied onto the support section TA on a top side OS, which faces away from an underside US of the implant I, on which the implant I is fixated to bone.

The cover layer DS consists of ultra high molecular weight polyethylene (UHMWPE) in the present example case, which, as a biocompatible material, has a lower hardness than the polyether ether ketone (PEEK) of the support section TA. The cover layer DS in the implant I is plate-shaped and porous, wherein, on the top side OS of the implant I, the support section TA is embedded into the cover layer DS. Due to the porous design of the cover layer DS, a particularly soft surface is created on the top side OS of the implant I here, wherein an ingrowth of tissue can also take place and bodily fluids can pass through. This also enables vascularization to take place in the region of the implant I and soft tissue to be better retained.

For manufacturing the implant I, the support section TA of the lattice structure GS, after having been additively manufactured, was subjected to a plasma treatment/plasma activation for cleaning purposes, wherein the support section TA was then placed together with polyethylene granules layer-by-layer in a negative mold. The implant I was then formed by heating the polyethylene together with the polyether ether ketone within the framework of a pressing process and, during the course thereof, the polyethylene was fused with the polyether ether ketone.

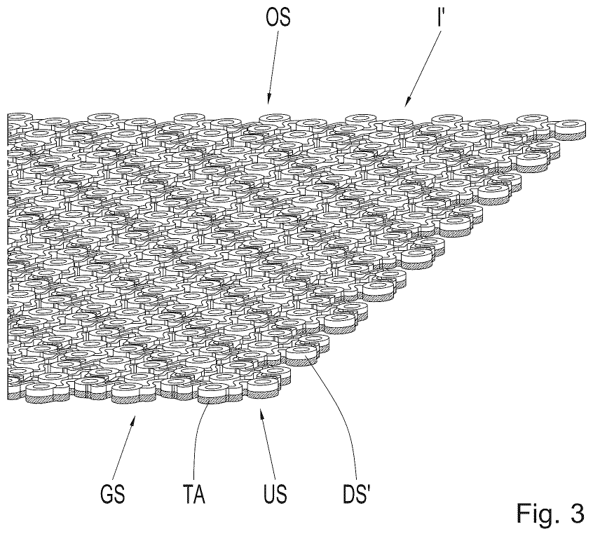

FIG. 3 shows a perspective view of an implant I′ according to a second possible example embodiment of the invention. This implant I′ largely corresponds to the preceding example variant according to FIGS. 1 and 2. The implant I′ also has a lattice structure GS, which is formed by a support section TA, which is made of polyether ether ketone (PEEK), has a mesh structure, and has been additively manufactured.

Furthermore, the support section TA is also coated in sections in the implant I′, in that a cover layer DS' has been applied on the support section TA on a top side OS of the implant I′. The cover layer DS′, in conformance with the example variant according to FIGS. 1 and 2, is made of polyethylene (PE), wherein, in contrast to the implant I from FIGS. 1 and 2, the cover layer DS' in this case is not porous, for the purpose of which polyethylene powder was used instead of polyethylene granules to manufacture the implant I′. Compared to the implant I from FIGS. 1 and 2, a smooth surface of the implant I′ on the top side OS is thereby achieved. As is also apparent in FIG. 3, the cover layer DS' is designed with a structuring which corresponds to the mesh structure of the underlying support section TA. As a result, even with the non-porous design of the cover layer DS′, an ingrowth of tissue and passage of bodily fluids is made possible. For the rest, the implant I′ corresponds to the implant I from FIGS. 1 and 2, such that reference is made to the descriptions thereof.

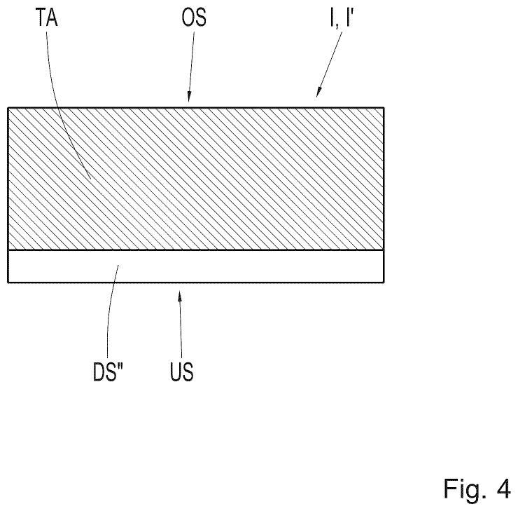

FIGS. 4 through 6 each show possible example modifications of the two implants I and I′ according to FIGS. 1 through 3. The support section TA of the respective implant I or I′ in the possible example modification according to FIG. 2 is partially coated due to the fact that, in this case, a cover layer DS″ is provided on the underside US of the implant I and I′. This cover layer DS″ is again equipped with a lower hardness than the support section TA by forming the cover layer DS″ from polyethylene (PE). In addition, the cover layer DS″, analogously to the one described above, can be porous and plate-shaped or non-porous and provided with a structuring corresponding to the mesh structure of the support section TA.

In the possible example modification according to FIG. 5, the implant I or I′, by contrast, is coated on its support section TA both on the top side OS with the cover layer DS or DS′, respectively, and on the underside US with the coating layer DS″, whereby the support section TA is sandwiched between the cover layers DS or DS′, respectively, and DS″.

FIG. 6 shows a possible example modification of the two implants I and I′ according to FIGS. 1 and 3, wherein the support section TA is completely encapsulated in the cover layer DS′″ in this case. In principle, this cover layer DS′″ could also be porous or non-porous.

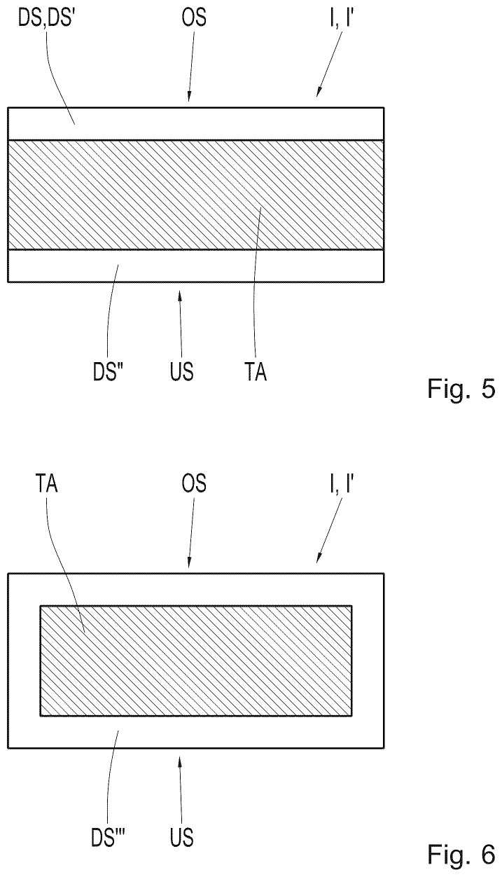

FIG. 7 shows a schematic view of an implant I″ according to a further example embodiment of the invention, wherein this implant I″ substantially corresponds to the implant I′ from FIG. 3. In contrast to the implant I′, in the implant I″, a lattice structure GS' is not formed in this case by a single support section, but rather by a plurality of support sections TA1 and TA2, each of which has a mesh structure. The respective mesh structure of the respective support section TA1 or TA2 is formed by ring-shaped segments S again, which form through-holes DO for bone screws and are connected to one another via intermediate segments ZS.

The support sections TA1 and TA2 are connected to one another via a respective intermediate connecting section VA to form the lattice structure GS′, wherein this connecting section VA is designed with a lower rigidity than the support sections TA1 and TA2. Thus, a higher flexibility of the implant I″ compared to the implant I′ from FIG. 3 is achieved.

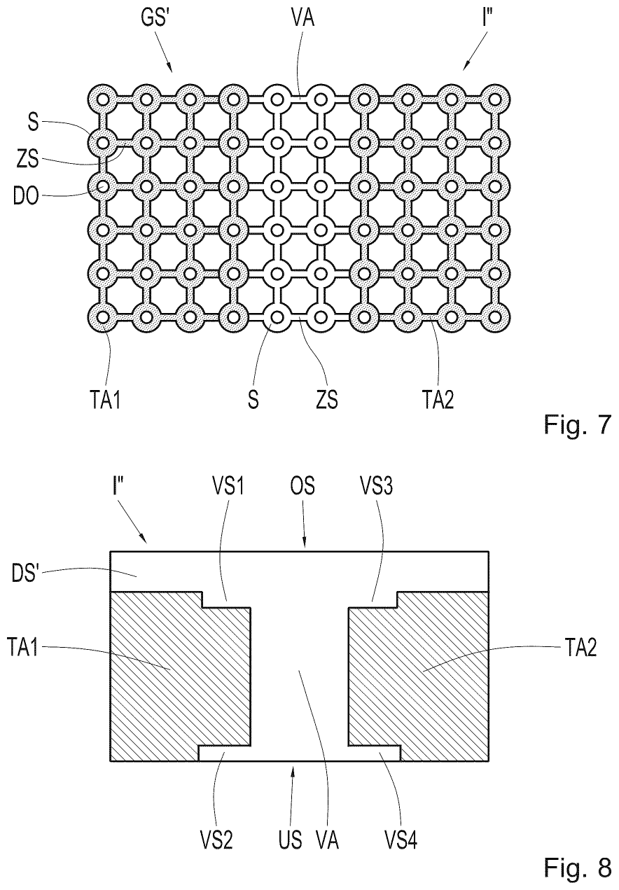

The connecting section VA, in conformance with the support sections TA1 and TA2, also has a mesh structure composed of segments S and intermediate segments ZS, wherein the lower rigidity is achieved by the fact that the connecting section VA is formed from polyethylene (PE). During the course of the production of the implant I″, an integral bond was established between the respective support section TA1 or TA2 and the intermediate connecting section VA in the pressing process. In order to further increase the load-bearing capacity of the respective connection, the intermediate connecting section VA covers the respective support section TA1 or TA2 in the region of the respective connection in each case, as is apparent in the schematic view of the implant I″ in FIG. 8. For this purpose, the connecting section VA is equipped with the connecting segments VS1 through VS4, which protrude from the connecting section VA in a direction extending transversely to the top side OS and the underside US and with which the connecting section VA engages around the respective support section TA1 or TA2 on each side in the respective connecting region.

Analogously to the example variant according to FIG. 3, a cover layer DS' is also provided, with which, in the implant I″, the support sections TA1 and TA2 and also the connecting section VA are coated on the top side OS in this case. The cover layer DS' is designed as a non-porous cover layer. For the rest, the implant I″ corresponds to the example variant according to FIG. 3, such that reference is made to the description thereof. The modifications according to FIGS. 4 through 6 could also be implemented in the implant I″.

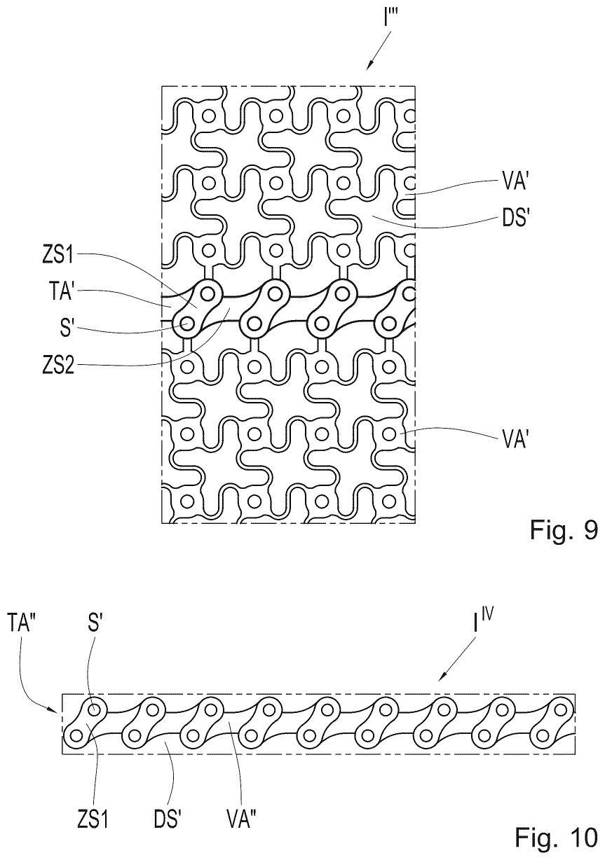

FIG. 9 shows a part of a further possible example embodiment of an implant′″, which largely corresponds to the preceding example variant according to FIGS. 7 and 8. This implant I′″ also includes a plurality of support sections TA′, only one support section TA′ of which can be seen in FIG. 9, however. In contrast to the implant I″ from FIGS. 7 and 8, the support section TA′ is formed in a plate-shaped and rigid manner from polyether ether ketone (PEEK). Ring-shaped segments S′ are connected to one another in pairs via intermediate segments ZS1 and, as a result, form figure eight-shaped structures, wherein these structures are then connected to one another via further, web-like intermediate segments ZS2 so as to form the support section TA′.

The support section TA′ is also connected to adjacent support sections via intermediate connecting sections VA′, each of which consists of polyethylene (PE). A connection of the individual connecting section VA′ to the respective support section TA′ is achieved in a manner similar to the example variant according to FIGS. 7 and 8. In addition, the support sections TA′ and the connecting sections VA′ are also provided with a cover layer DS′ in the implant′″, wherein further modifications are also conceivable within the scope of one of the example variants according to FIGS. 4 through 6. For the rest, the example embodiment according to FIG. 9 corresponds to the example variant according to FIGS. 7 and 8, such that reference is made to the descriptions thereof.

Finally, FIG. 10 shows an example embodiment of an implant I^{IV}, wherein this implant I^{IV} largely corresponds to the preceding example variant according to FIG. 9. The difference is that support sections TA″ in the implant I^{IV} are formed from polyether ether ketone (PEEK) as figure eight-shaped structures, in each of which ring-shaped segments S′ are connected to one another in pairs via an intermediate segment ZS1. The support sections TA″ are then connected to one another via connecting sections VA″, each of which has a lower rigidity compared to the support sections TA″, in that these connecting sections VA″ consist of polyethylene (PE). A connection of the individual connecting section VA″ to the respective support section TA″ is carried out in a manner similar to the example variant according to FIGS. 7 and 8, wherein the support sections TA″ and the connecting sections VA″ are also coated with a cover layer DS′. In this case as well, a further example modification can also be achieved within the scope of one of the example variants according to FIGS. 4 through 6.

By the example embodiments according to example aspects of the invention, an implant can be created in each case for an areal treatment of bone defects with low manufacturing outlay, wherein, when this implant is used, minor tissue irritations in the body of the patient are induced.

Modifications and variations can be made to the embodiments illustrated or described herein without departing from the scope and spirit of the invention as set forth in the appended claims. In the claims, reference characters corresponding to elements recited in the detailed description and the drawings may be recited. Such reference characters are enclosed within parentheses and are provided as an aid for reference to example embodiments described in the detailed description and the drawings. Such reference characters are provided for convenience only and have no effect on the scope of the claims. In particular, such reference characters are not intended to limit the claims to the particular example embodiments described in the detailed description and the drawings.

List of reference characters

| | |
|---|---|
| I, I′, I″, I′″, I^{IV} | implant |
| GS, GS′ | lattice structure |
| TA, TA1, TA2, TA′, TA″ | support section |
| S, S′ | segment |
| ZS, ZS1, ZS2 | intermediate segment |
| DO | through-hole |
| DS, DS′, DS″, DS′″ | cover layer |
| OS | top side |
| US | underside |
| VA, VA′, VA″ | connecting section |
| VS1, VS2, VS3, VS4 | connecting segment |

1-15: (canceled)

16. An implant (I; I′; I″; I′″; IIV) for an areal treatment of a bone defect, comprising:
a flexible lattice structure (GS; GS′) having a top side (OS) and an underside (US) facing away from the top side (OS), the lattice structure (GS; GS′) fixable to bone at the underside (US) of the lattice structure (GS; GS′), the lattice structure (GS; GS′) comprising at least one support section (TA; TA1, TA2; TA′; TA″),
wherein the at least one support section (TA; TA1, TA2; TA′; TA″) has been produced by an additive manufacturing process, and
wherein a cover layer (DS; DS′; DS″; DS′″) is at least partially applied on the at least one support section (TA; TA1, TA2; TA′; TA″), the cover layer having a hardness that is less than a hardness of the at least one support section (TA; TA1, TA2; TA′; TA″).

17. The implant (I; I′; I″; I′″; IIV) of claim 16, wherein the at least one support section (TA; TA1, TA2; TA′; TA″) is coated with the cover layer (DS; DS′; DS′″) on at least one subsection of the top side (OS) of the lattice structure (GS; GS′) or completely on the top side (OS) of the lattice structure (GS; GS′).

18. The implant (I; I′; I″; I′″; IIV) of claim 16, wherein the at least one support section (TA; TA1, TA2; TA′; TA′″) is coated with the cover layer (DS″; DS′″) on at least one subsection of the underside (US) of the lattice structure (GS; GS′) or completely on the underside (US) of the lattice structure (GS; GS′).

19. The implant (I; I′; I″; I′″; IIV) of claim 16, wherein the at least one support section (TA; TA1, TA2; TA′; TA″) is completely encapsulated in the cover layer (DS′″).

20. The implant (I; I′; I″) of claim 16, wherein the at least one support section (TA; TA1, TA2) has a mesh structure formed by closed segments(S), and the closed segments(S) are connected by intermediate segments (ZS).

21. The implant (I″; IIV) of claim 16, wherein the at least one support section (TA′; TA″) is plate-shaped.

22. The implant (I; I″; I′; IIV) of claim 16, wherein the cover layer (DS; DS″; DS′″) is porous.

23. The implant (I; I″; I′″; IIV) of claim 22, wherein the at least one support section (TA; TA1, TA2; TA′; TA″) is at least partially embedded into the porous cover layer (DS; DS″; DS′″).

24. The implant (I′; I″; I′″; IIV) of claim 16, wherein the cover layer (DS′; DS″; DS′″) is non-porous.

25. The implant (I′; I″; I′″; IIV) of claim 24, wherein a structuring of the cover layer (DS′; DS″; DS′″) corresponds to a structuring of the at least one support section (TA; TA1, TA2; TA′; TA″) in a region of the coating with each cover layer (DS′; DS″; DS′″).

26. The implant (I; I′; I″; I′″; IIV) of claim 16, wherein the at least one support section (TA; TA1, TA2; TA′; TA″) is formed of a first biocompatible material, the cover layer (DS; DS′; DS″; DS′″) is formed a second biocompatible material, and a hardness of the second biocompatible material is less than a hardness of the first biocompatible material.

27. The implant of claim 26, wherein the first biocompatible material is a metal or a metal alloy.

28. The implant of claim 27, wherein the first biocompatible material is titanium or a titanium alloy.

29. The implant of claim 26, wherein the first biocompatible material is a plastic.

30. The implant of claim 29, wherein the first biocompatible material is a thermoplastic.

31. The implant of claim 29, wherein the first biocompatible material is polyether ether ketone.

32. The implant of claim 26, wherein the second biocompatible material is a plastic.

33. The implant of claim 32, wherein the second biocompatible material is polyethylene.

34. The implant (I; I′) of claim 16, wherein the lattice structure (GS) has one single continuous support section (TA).

35. The implant (I″; I′″; IIV) of claim 16, wherein the lattice structure (GS′) comprises a plurality of support sections (TA1, TA2; TA′; TA″), wherein adjacent support sections of the plurality of support sections (TA1, TA2; TA′; TA″) are connected via respective intermediate connecting sections (VA, VA′, VA″), a rigidity of the intermediate connecting sections (VA, VA′, VA″), being less than a rigidity of the adjacent support sections.
